(19) United States
(12) Patent Application Publication
(10) Pub. No.: US 2018/0042664 A1
(43) Pub. Date: Feb. 15, 2018

(54) STEERABLE RADIOFREQUENCY DENERVATION PROBE FOR GENICULAR NERVE DENERVATION AND METHODS OF USE

(71) Applicant: Pacestter, Inc., Sylmar, CA (US)

(72) Inventor: Conrad Juergens, Etterbeek (BE)

(21) Appl. No.: 15/233,782

(22) Filed: Aug. 10, 2016

(51) Int. Cl. A61B 18/14; A61B 6/12

(52) U.S. Cl. CPC A61B 18/148; A61B 6/12; A61B 18/1477; A61B 2018/00434

(57) ABSTRACT

The present disclosure provides a radiofrequency genicular nerve denervation probe and methods of using the probe to address chronic knee pain by producing therapeutic lesions or tissue alterations. In many embodiments, the denervation probe is a steerable, multi-electrode probe that includes one or more injection ports that allow one or more fluids to be injected at the target site before, during, or after a denervation procedure. The denervation probe may also include in many embodiments one or more thermocouples configured to provide targeted temperature readings. The denervation probe has the capability to perform a customized or preset sequence of lesions to provide desirable procedural flexibility.

### A. FIELD OF THE DISCLOSURE

The present disclosure relates generally to methods and apparatuses for denervation of the genicular nerves to reduce chronic pain in the knee. In particular, the present disclosure relates to a steerable radiofrequency denervation probe for use in genicular nerve denervation wherein the denervation probe is steerable such that it bends at a desired point in a uni-directional manner on the distal portion thereof to allow for improved genicular nerve denervation.

### B. BACKGROUND

Chronic painfil knee osteoarthritis and related conditions are major causes of disability in many older adults. In many cases, chronic osteoarthritis is not effectively managed with current non-pharmacological or pharmacological treatments. In these cases, radiofrequency neurotomy may be a therapeutic alternative for chronic pain.

Radiofrequency neurotomy specifically targets the genicular nerves (the Medial Superior Genicular, the Lateral Superior Genicular, and the Inferomedial Genicular) of the knee. To date, the radiofrequency denervation treatment of these nerves has been carried out utilizing either a standard radiofrequency needle or a cooled radiofrequency needle/probe. Generally, the active tip of the device is placed near the epicondyle to capture the nerve in its suspected location. Either a straight or a curved active tip has been used in past procedures.

Although radiofrequency neurotomy provides a promising treatment for managing chronic painful knee osteoarthritis and other painful knee conditions, improvements in the radiofrequency probes or needles utilized in the procedure are desirable to improve the effectiveness of lesions made in the tissue, as well as monitor the temperatures created in the tissue during the procedures. Additionally, it would be desirable to provide the radiofrequency probes or needles with one or more injection ports therein to allow for the delivery of one or more fluids before, during, or after a procedure.

### BRIEF SUMMARY OF THE DISCLOSURE

The present disclosure provides a radiofrequency genicular nerve denervation probe and methods of using the probe to address chronic knee pain by producing therapeutic lesions or tissue alterations on the genicular nerves. In many embodiments, the denervation probe is a steerable, multi-electrode probe that includes one or more injection ports that allow one or more fluids to be injected at the target site before, during, or after a denervation procedure. The denervation probe may also include in many embodiments one or more thermocouples configured to provide targeted temperature readings. The denervation probe has the capability to perform a customized or preset sequence of lesions to provide desirable procedural flexibility. Various alternative embodiments of the present disclosure are set forth herein.

In one embodiment, the present disclosure is directed to a method of treating chronic pain in the knee. The method comprises (i) puncturing a target site of a patient with a distal portion of a radiofrequency denervation probe to allow access to at least one genicular nerve; (ii) advancing the radiofrequency denervation probe towards the at least one genicular nerve; (iii) activating a steering mechanism of the radiofrequency denervation probe to cause a distal portion of a shaft of the radiofrequency denervation probe to bend in a desired direction; (iv) advancing the bent radiofrequency denervation probe adjacent to the at least one genicular nerve; and (v) creating one or more lesions on the at least one genicular nerve, wherein the distal portion of the shaft of the radiofrequency denervation probe includes at least one ablation electrode.

In another embodiment, the present disclosure is directed to a method of reducing the pain associated with chronic knee osteoarthritis. The method comprises (i) puncturing a target site of a patient with a distal portion of a radiofrequency denervation probe to allow access to the medial superior genicular nerve; (ii) advancing the radiofrequency denervation probe towards the medial superior genicular nerve; (iii) activating a steering mechanism of the radiofrequency denervation probe to cause a distal portion of a shaft of the radiofrequency denervation probe to bend in a desired direction; (iv) advancing the bent radiofrequency denervation probe adjacent to the medial superior genicular nerve; and (v) creating one or more lesions on the medial superior genicular nerve, wherein the distal portion of the shaft of the radiofrequency denervation probe includes at least two ablation electrodes, at least two injection ports, and at least two thermocouples.

In another embodiment, the present disclosure is directed to a genicular nerve denervation probe. The genicular nerve denervation probe comprises (i) a handle including a steering mechanism; (ii) a catheter shaft having proximal portion and a distal portion, wherein the distal portion is configured for initial percutaneous advancement, and wherein the catheter shaft is configured to allow the distal portion to bend uni-directionally at a predetermined position using the steering mechanism; and (iii) at least one ablation electrode located on the distal portion of the catheter shaft.

In another embodiment, the present disclosure is directed to a method of treating chronic pain in the knee. The method comprises: (i) puncturing a target site of a patient with a distal portion of a radiofrequency denervation probe to allow access to at least one genicular nerve; (ii) advancing the radiofrequency denervation probe towards the at least one genicular nerve; (iii) activating a steering mechanism of the radiofrequency denervation probe to cause a distal portion of a shaft of the radiofrequency denervation probe to bend in a desired direction; wherein the distal portion of the shaft of the radiofrequency denervation probe includes at least a proximal directional ablation electrode and a distal directional ablation electrode with respect to the bend; (iv) advancing the bent radiofrequency denervation probe adjacent to the at least one genicular nerve; (v) creating a first bipolar lesion using the proximal directional ablation electrode and the distal directional ablation electrode simultaneously; (vi) creating a second monopolar lesion using the proximal directional ablation electrode; and (vii) creating a third monopolar lesion using the distal directional ablation electrode.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIG. 1 is a diagrammatic view of a genicular nerve denervation system including a genicular nerve denervation probe as described herein.

FIG. 2 is a front view of a genicular nerve denervation probe of one embodiment of the present disclosure.

FIG. 3 is a side view of the genicular nerve denervation probe of FIG. 2 in an unbent conformation.

FIG. 4 is a side view of the genicular nerve denervation probe of FIG. 2 in a bent conformation.

FIG. 5 is a flow chart of one embodiment of a method of the present disclosure for treating chronic pain in the knee.

FIG. 6 is a flow chart of one embodiment of a method of the present disclosure for reducing the pain associated with chronic knee osteoarthritis.

FIGS. 7a and 7b are flow charts of one embodiment of a method of denervating three branches of the genicular nerve for knee joint intervention.

FIG. 8 is a side view of the genicular nerve denervation probe of FIG. 2 to show internal construction.

FIG. 9 is the genicular nerve denervation probe of FIG. 8 in a bent conformation.

Corresponding reference characters indicate corresponding parts throughout the several views of the drawings. It is understood that that Figures are not necessarily to scale.

### DETAILED DESCRIPTION OF THE DISCLOSURE

The present disclosure provides methods, systems and apparatuses for genicular nerve denervation. In many embodiments, a steerable genicular nerve denervation probe is disclosed that provides numerous advantages over conventional genicular denervation probes and/or needles as further described herein. A number of methods for genicular nerve denervation utilizing the genicular nerve denervation probe of the present disclosure are also provided.

More specifically, many embodiments of the present disclosure provide a genicular nerve denervation probe that is steerable (that is, bendable in a uni-directional manner) and includes one or multiple electrodes positioned thereon that can provide directional active heating (directional electrodes located on one side of the probe) or treatment zones to allow for the controlled heating of only the target zone (i.e., one or more genicular nerves) within the knee. This combination of steerability and directional heating using multiple electrodes allows the denervation probe to better create the desired lesions to the contour of the treatment area/anatomical site within the knee. With this improved contoured lesion, more of the targeted nerve site is denervated and less unwanted damage to the surrounding tissue occurs. Additionally, the denervation probes of the present disclosure include one or more injections sites or ports thereon at or near the active electrodes such that there is an increased opportunity for the adequate spread of any injected fluids along the active portion of the denervation probe. In many embodiments, the denervation probe will also include one or more thermocouples integrated into the active portions of the denervation probe to provide highly accurate and targeted temperature readings at the lesion formation sites to further improve outcomes.

The genicular nerve denervation probes as described herein are in many embodiments powered by a radiofrequency generator that can be programmed to allow the denervation probe to perform preset and/or customized lesion formations within the knee to provide for improved procedural flexibility. Before or during a denervation procedure, multiple variables including time, temperature, bipolar lesions, monopolar lesions etc. can be controlled and changed as needed to improve the performance and overall result of the denervation procedure.

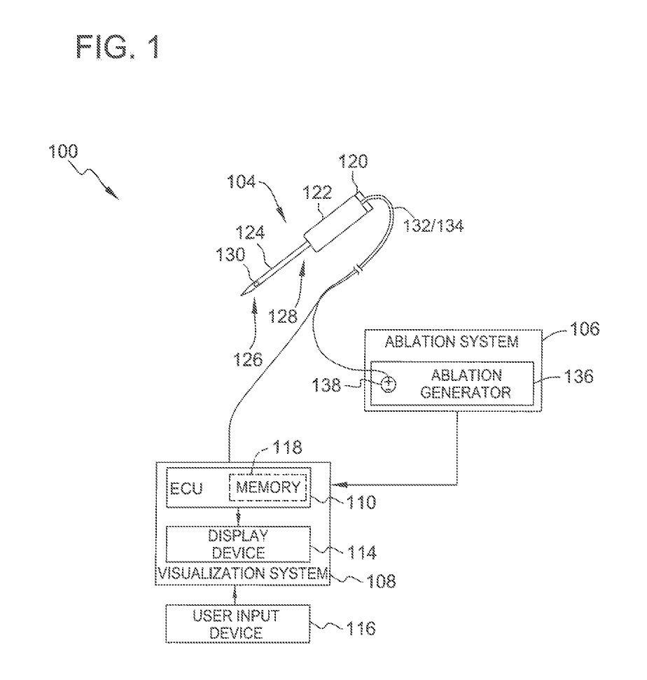

Referring now to the Figures, FIG. 1 illustrates one exemplary embodiment of a genicular nerve denervation system 100 for creating one or more lesions on or near one or more of the genicular nerves. System 100 includes genicular nerve denervation probe 104 of the present disclosure. System 100 additionally includes ablation system 106, and system 108 for the visualization, navigation, and/or mapping of internal body structures, such as the knee. System 108 may include, for example and without limitation, an electronic control unit (ECU) 110, display device 114, user input device 116, and memory 118. Alternatively, ECU 110 and/or display device 114 may be separate and distinct from, but electrically connected to and configured for communication with, system 108. As will be recognized by one skilled in the art based on the disclosure herein, other systems and configurations could also be used within the scope of the present disclosure.

With continued reference to FIG. 1, genicular nerve denervation probe 104 is provided for the denervation of internal body tissues, such as one or more of the genicular nerves in and around the knee. In an exemplary embodiment, genicular nerve denervation probe 104 comprises a radio frequency (RF) ablation denervation probe. It should be understood, however, that genicular nerve denervation probe is not limited to an RF ablation denervation probe.

In an exemplary embodiment, genicular nerve denervation probe 104 is electrically connected to ablation system 106 to allow for the delivery of RF energy. Genicular nerve denervation probe 104 may include a cable connector or interface 120, handle 122, shaft 124 having a distal end 126 and proximal end 128 (as used herein, “proximal” refers to a direction toward the end of genicular nerve denervation probe near the operator, and “distal” refers to a direction away from the operator and (generally) inside the body of a subject or patient), and one or more electrodes 130 mounted in or on shaft 124 of genicular nerve denervation probe 104. In an exemplary embodiment, electrode 130 is disposed at or near distal end 126 of shaft 124, with electrode 130 comprising an ablation electrode. Genicular nerve denervation probe 104 may further include other conventional components such as, for example and without limitation, sensors, additional electrodes, thermocouples and corresponding conductors or leads, or additional ablation elements, e.g., a high intensity focused ultrasound ablation element and the like.

Connector 120 provides mechanical and electrical connection(s) for cables 132 and 134 extending from ablation system 106, and visualization, navigation, and/or mapping system 108. Connector 120 is conventional in the art and is disposed at the proximal end of genicular nerve denervation probe 104. Handle 122 provides a location for the operator to hold genicular nerve denervation probe 104 and may further provide means for steering or guiding shaft 124 as further described herein within the knee.

Shaft 124 is generally an elongated, tubular, partially flexible member configured for placement and movement within the knee. Shaft 124 supports, for example and without limitation, electrode 130, associated conductors and thermocouples, and possibly additional electronics used for signal processing or conditioning. Shaft 124 may also permit transport, delivery and/or removal of fluids (including irrigation fluids, cryogenic ablation fluids, and bodily fluids), medicines, and/or surgical tools or instruments.

With further reference to FIG. 1, ablation system 106 is comprised of, for example, ablation generator 136. Ablation generator 136 generates, delivers, and controls RF energy output by genicular nerve denervation probe 104 and electrode 130 thereof, in particular. In an exemplary embodiment, ablation generator 136 includes RF ablation signal source 138 configured to generate an ablation signal that is output across a pair of source connectors: a positive polarity connector SOURCE (+), which may be electrically connected to electrode 130 of genicular nerve denervation probe 104; and a negative polarity connector SOURCE (−). It should be understood that the term connectors as used herein does not imply a particular type of physical interface mechanism, but is rather broadly contemplated to represent one or more electrical nodes. Source 138 is configured to generate a signal at a predetermined frequency in accordance with one or more user specified parameters (e.g., power, time, etc.) and under the control of various feedback sensing and control circuitry as is known in the art. Source 138 may generate a signal, for example, with a frequency of about 450 kHz or greater. Ablation generator 136 may also monitor various parameters associated with the ablation procedure including, for example, impedance, the temperature at the distal tip of the genicular nerve denervation probe, applied ablation energy, and the position of the catheter, and provide feedback to the clinician or another component within system 100 regarding these parameters. One RF generator suitable for use in system 100 is set forth and described in U.S. Pat. No. 8,818,503.

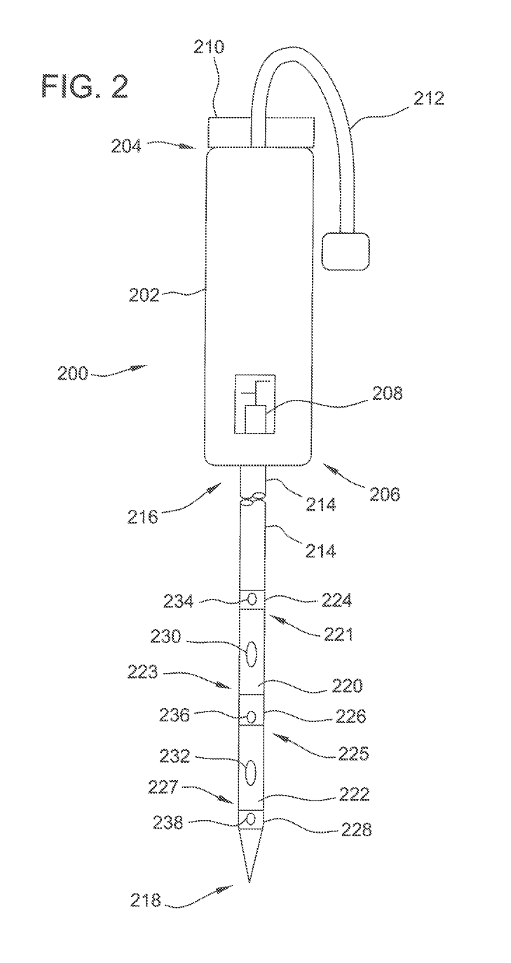

Referring now to FIG. 2, there is illustrated a front view of a genicular nerve denervation probe 200 in accordance with one embodiment of the present disclosure. Genicular nerve denervation probe 200 is suitable for use in the denervation of the genicular nerves of the knee, as described herein. Genicular nerve denervation probe 200 includes handle 202 including proximal end 204 and distal end 206. Handle 202 houses switch 208 that may be sized and configured to be slid into one or two or more positioning slots (not shown) and used to control the amount of steering and bending of genicular nerve denervation probe 200 as further described herein. Because genicular nerve denervation probe 200 is steerable, it's exact position within the knee (and more particularly the exact position of the electrodes located thereon as described below) can be precisely controlled to allow the opportunity to create one or more lesions conforming to the contour of the desired anatomical area (treatment site). This is highly advantageous as the genicular nerves tend to be positioned on and/or near the bones of the knee, making access thereto potentially challenging in many cases. Near proximal end 204 of handle 202 is connector port 210 for connecting genicular nerve denervation probe 200 to an RF generator (not shown in FIG. 2 but see FIG. 1). Also shown in FIG. 2 is electrical connection cable 212 that may optionally include one or more tubing structures (not shown) for the introduction of one or more liquids into genicular nerve denervation probe 200 for distribution at the target site of denervation.

FIG. 2 additionally shows shaft 214 of genicular nerve denervation probe 200 that may optionally be insulated in some embodiments as further described herein. Shaft 214 includes a proximal end 216 and a distal end 218. Distal end 218 is illustrated in FIG. 2 as a non-RF-active sharpened tip to allow for tissue penetration and access to a target site. Of course, as will be recognized by one skilled in the art based on the disclosure herein, distal end 218 may or may not be RF-active and may have a different shape other than a sharpened tip in other embodiments. For example, in another embodiment the distal end may be blunt such that any puncturing of a target site is done with another instrument. Shaft 214 additionally includes a first electrode 220 located near proximal end 216 of shaft 214 and a second electrode 222 located near distal end 218 of shaft 214. First electrode 220 has a proximal end 221 and a distal end 223 and second electrode 222 has a proximal end 225 and a distal end 227. Within the scope of the present disclosure, first electrode 220 and second electrode 222 may be energized to create a desired lesion independently, or may be energized together simultaneously. Both monopolar lesions and dual lesions are within the scope of the present disclosure and the electrodes may be used in some embodiments with a grounding pad. Although illustrated in FIG. 2 with a first electrode 220 and a second electrode 222, the genicular nerve denervation probes of the present disclosure may have one, two three, four, five or more electrodes in accordance with other embodiments of the present disclosure.

With continued reference to FIG. 2, shaft 214 additionally includes a first radiopaque insulated marker 224 that may be used to optically selectively identify proximal end 221 of first electrode 220 during a procedure, a second radiopaque insulated marker 226 that may be used to optically selectively identify the separation between first electrode 220 and second electrode 222, and a third radiopaque insulated marker 228 that may be used to optically selectively identify distal end 227 of second electrode 222 during a procedure. In another embodiment, more or less radiopaque insulated markers may be used. Shaft 214 additionally includes proximal electrode thermocouple 230 and distal electrode thermocouple 232. By including multiple thermocouples on genicular nerve denervation probe 200 and integrating them into the electrodes present, there is provided highly accurate and targeted temperature readings throughout a procedure such that the creation of the lesions in the one or more genicular nerves may be constantly controlled and evaluated throughout the procedure. Also illustrated in FIG. 2 is proximal injection port 234, medial injection port 236, and distal injection port 238. These multiple injection ports near first electrode 220 and second electrode 222 provide for enhancing spreading and coverage of injected fluids (saline solution, anesthetics, steroids, other medicines, etc.) along the active portions of genicular nerve denervation probe 200 such that any injected fluids may reach the desired areas along the treatment area within the knee. An operator may choose to use one, two, or all three injection ports during a procedure. More or less injection ports may also be used with genicular nerve denervation probe 200 in accordance with the present disclosure.

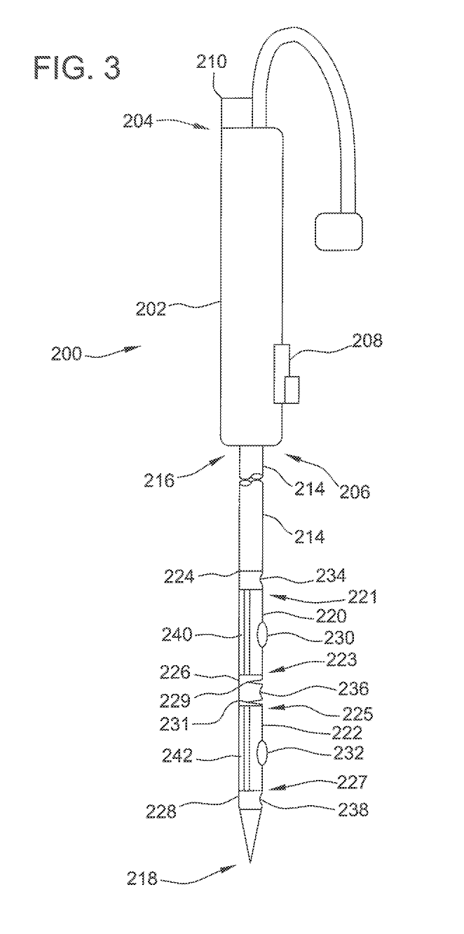

Referring now to FIG. 3, there is shown a side view of the genicular nerve denervation probe of FIG. 2 in an unbent conformation. FIG. 3 shows genicular nerve denervation probe 200 including handle 202 having proximal end 204 and distal end 206. Handle 202 includes switch 208. Near proximal end 204 of handle 202 is connector port 210. Shaft 214 includes a proximal end 216 and a distal end 218. Shaft 214 additionally includes a first electrode 220 located near proximal end 216 of shaft 214 and a second electrode 222 located near distal end 218 of shaft 214. First electrode 220 has a proximal end 221 and a distal end 223 and second electrode 222 has a proximal end 225 and a distal end 227.

With continued reference to FIG. 3, shaft 214 includes a first radiopaque insulated marker 224, a second radiopaque insulated marker 226, and a third radiopaque insulated marker 228. Second radiopaque insulated marker 226 includes proximal bend joint 229 and distal bend joint 231 to all for bending as described herein. Shaft 214 additionally includes proximal electrode thermocouple 230 and distal electrode thermocouple 232. Also illustrated in FIG. 3 are proximal injection port 234, medial injection port 236, and distal injection port 238. Although illustrated in FIG. 3 as having three injection ports, it is within the scope of the present disclosure for genicular nerve denervation probe 200 to include only one, or even only two injection ports. In some embodiments, genicular nerve denervation probe 200 may include no injection ports. FIG. 3 also shows proximal insulation 240 located behind first electrode 220 and distal insulation 242 located behind second electrode 222. Proximal insulation 240 and distal insulation 242 allow for directional active heating or directional active treatment zones of each of first electrode 220 and second electrode 222. This directional active heating provides targeted and controlled heating and energy (RF energy, for example) placement into only a desired area with a clear direction of treatment; that is, proximal insulation 240 and distal insulation 242 allow RF energy (or another type of energy) to flow or be directed only in a single targeted direction such that tissue within a body can be more carefully treated and lesions created without disturbing surrounding tissue.

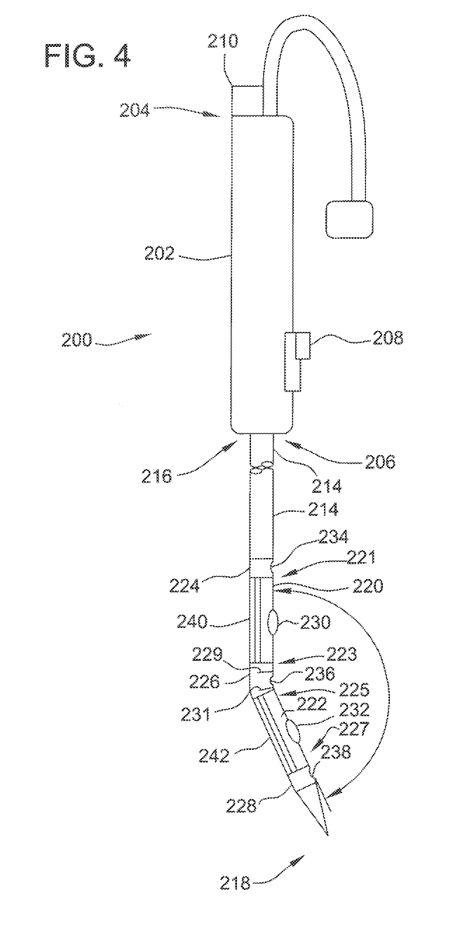

Referring now to FIG. 4, there is shown a side view of the genicular nerve denervation probe of FIG. 2 in a bent conformation. FIG. 4 shows genicular nerve denervation probe 200 including handle 202 having proximal end 204 and distal end 206. Handle 202 includes switch 208. Near proximal end 204 of handle 202 is connector port 210. Shaft 214 includes a proximal end 216 and a distal end 218. Shaft 214 additionally includes a first electrode 220 located near proximal end 216 of shaft 214 and a second electrode 222 located near distal end 218 of shaft 214. First electrode 220 has a proximal end 221 and a distal end 223 and second electrode 222 has a proximal end 225 and a distal end 227.

With continued reference to FIG. 4, shaft 214 includes a first radiopaque insulated marker 224, a second radiopaque insulated marker 226, and a third radiopaque insulated marker 228. Second radiopaque insulated marker 226 includes proximal bend joint 229 and distal bend joint 231. Shaft 214 additionally includes proximal electrode thermocouple 230 and distal electrode thermocouple 232. Also illustrated in FIG. 3 are proximal injection port 234, medial injection port 236, and distal injection port 238. FIG. 4 also shows proximal insulation 240 located behind first electrode 220 and distal insulation 242 located behind second electrode 222. As noted, FIG. 4 shows genicular nerve denervation probe 200 in a bent conformation; that is, genicular nerve denervation probe 200 is shown with proximal end 216 of shaft 214 misaligned with distal end 218 such that a curvature of shaft 214 is provided. This uni-directional curvature may be designed to fit desired embodiments for genicular nerve denervation, and in some embodiments the curvature may be from about 10 degrees to about 30 degrees, including from about 15 degrees to about 20 degrees. This curvature of shaft 214 of genicular nerve denervation probe 200 allows for the creation of a lesion that is more suitable to the curved bony structure along which the genicular nerves travel within the knee structure to provide an enhanced probability of fully denervating the desired genicular nerves. Additionally, the ability to adjust the conformation of the genicular nerve denervation probe from unbent to bent allows for easier placement of the probe and navigation within the knee structure thus limiting the potential for peripheral damage.

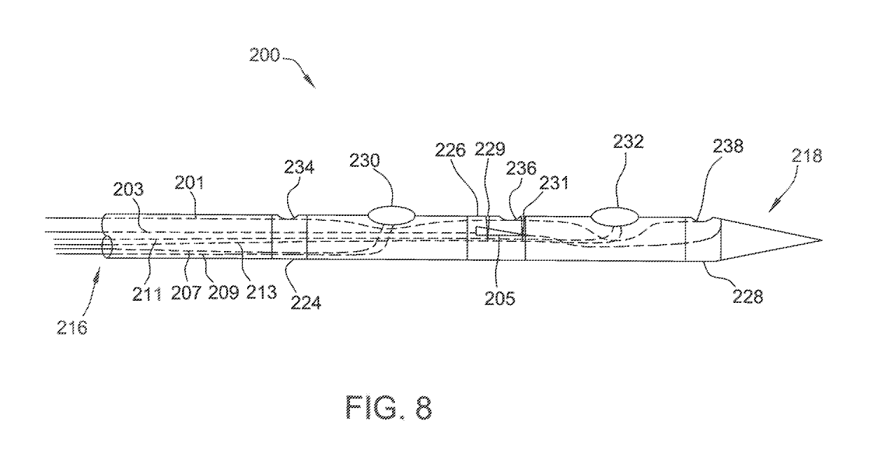

Referring now to FIG. 8, there is shown an additional side view of the genicular nerve denervation probe of FIG. 2 to further describe internal construction of the genicular nerve denervation probe. FIG. 8 shows genicular nerve denervation probe 200 having proximal end 216 and a distal end 218 and including proximal injection port 234, medial injection port 236, distal injection port 238, first radiopaque insulated marker 224, second radiopaque insulated marker 226, and third radiopaque insulated marker 228. Genicular nerve denervation probe 200 also includes to proximal electrode thermocouple 230, distal electrode thermocouple 232 and proximal bend joint 229 and distal bend joint 231. Also illustrated in FIG. 8 is injection tubing 201 connected to proximal injection port 234, medial injection port 236, and distal injection port 238. Injection tubing 201 may provide one or more liquids as desired to proximal injection port 234, medial injection port 236, and distal injection port 238 for use before, during, or after a procedure. FIG. 8 also shows steering cable 203 that is connected to switch 208 (not shown in FIG. 8 but see FIG. 4), steering lock device 205 (which is integrated into steering cable 203) and distal end 218 to allow for the steering and bending of genicular nerve denervation probe 200 as desired. Also shown is radiofrequency wire 207 and thermocouple wire 209 attached to proximal electrode thermocouple 230 and radiofrequency wire 211 and thermocouple wire 213 attached to distal electrode thermocouple 232.

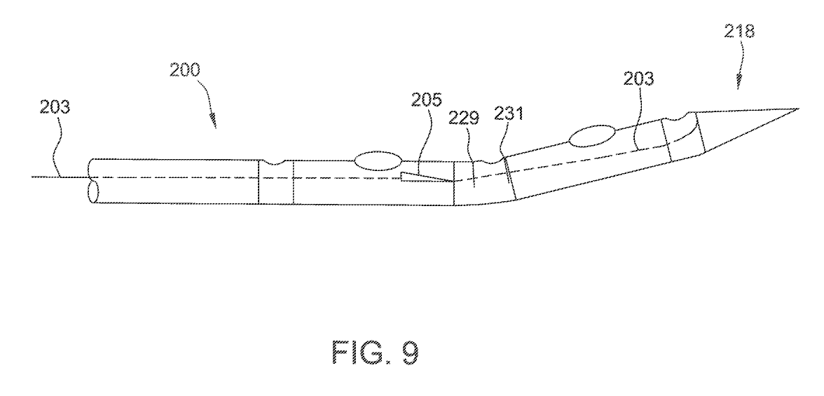

As illustrated in FIG. 8, genicular nerve denervation probe 200 includes steering cable 203 connected to switch 208 (not shown in FIG. 8 but see FIG. 4) to control the steering/bending of genicular nerve denervation probe 200, and specifically distal end 218 of genicular nerve denervation probe 200. Genicular nerve denervation probe 200 is naturally held in a straight conformation (unbent) by proximal bend joint 229 and distal bend joint 231, as well as steering lock device 205, which is integrated into steering cable 203 such that it moves therewith. As illustrated in FIG. 9, upon activation of switch 208 (see FIG. 4), steering cable 203 may be pulled proximally along with steering lock device 205 thus creating tension on distal end 218 causing bending at proximal bend joint 229 and distal bend joint 231 to create the desired level of curvature in genicular nerve denervation probe 200. Upon the release of switch 208 (see FIG. 4) the tension created on distal end 218 by steering cable 203 is released thus allowing the straight conformation of genicular nerve denervation probe 200 to be attained.

The steerable genicular denervation probes of the present disclosure are suitable for use in many genicular nerve denervation procedures to address chronic knee and related pain, including pain from chronic knee osteoarthritis and related conditions. Generally, the genicular denervation probe is inserted into the knee at a desired site and advanced until the distal portion of the probe has passed the lateral-most aspect of the bone adjacent to at least one genicular nerve. At this point, the steering mechanism of the probe as described herein is activated to enhance navigation within the knee structure and provide improved lesion creation due to the improved contour of the steerable probe against the bony structure. Any or all of the genicular nerves (i.e., the medial superior genicular, the lateral superior genicular, and the inferomedial genicular) may be targeted and denervated using methods of the present disclosure. One, two, and/or all three of the genicular nerves may be targeted in a single procedure. One or more lesions are created on the genicular nerves as described herein. The lesions created may be bipolar (without a grounding pad) and/or monopolar (with a grounding pad). In one specific embodiment, the probe includes two directional ablation electrodes located on opposite sides of the bend in the probe such that a series of lesions may be created. In this embodiment, a first bipolar lesion may be created (without a grounding pad) using both directional ablation electrodes simultaneously, followed by the creation of a second monopolar lesion using only one of the directional ablation electrodes (with a grounding pad), followed by the creation of a third monopolar lesion using only the second directional ablation electrode (with a grounding pad). In some embodiments, the genicular nerve denervation probe may be treated prior to puncture and insertion into the knee with a lubricating material to enhance the lubricious nature of the probe and improve its performance. Any suitable lubricating compound may be utilized on the probe including for example, a silicone-based material. In many embodiments, a silicone-based wipe may be used to improve the lubricous nature of the probe.

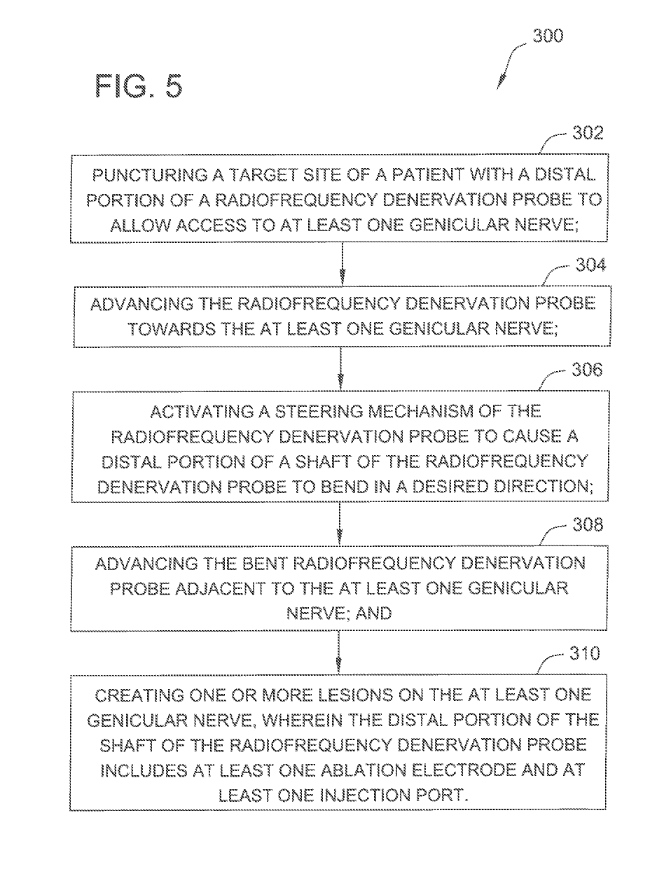

In one embodiment of the present disclosure, a method of treating chronic pain in the knee is disclosed. The method includes first puncturing a target site of a patient with a distal portion of a radiofrequency denervation probe to allow access to at least one genicular nerve. Although desirable in many embodiments to puncture the target site of the patient, such as a target site on or near the knee of the patient, with the denervation probe itself, in other embodiments the puncture may be done with another instrument. After the target site has been punctured, the denervation probe is advanced towards at least one genicular nerve and the steering mechanism of the denervation probe is activated to cause a distal portion of the denervation probe to bend in a desired direction to assist in navigation within the target site. The bent denervation probe is then advanced adjacent to at least one genicular nerve and one or more lesions are created on the genicular nerve to provide the desired therapy. In many embodiments, the denervation probe will include at least one injection port as described herein to allow one or more fluids to be introduced into the target site to improve the overall procedure. FIG. 5 is a flow chart of one embodiment of a method 300 for treating chronic pain in the knee. Method 300 includes puncturing 302 a target site of a patient with a distal portion of a radiofrequency denervation probe to allow access to at least one genicular nerve; advancing 304 the radiofrequency denervation probe towards the at least one genicular nerve; activating 306 a steering mechanism of the radiofrequency denervation probe to cause a distal portion of a shaft of the radiofrequency denervation probe to bend in a desired direction; advancing 308 the bent radiofrequency denervation probe adjacent to the at least one genicular nerve; and creating 310 one or more lesions on the at least one genicular nerve, wherein the distal portion of the shaft of the radiofrequency denervation probe includes at least one ablation electrode and at least one injection port.

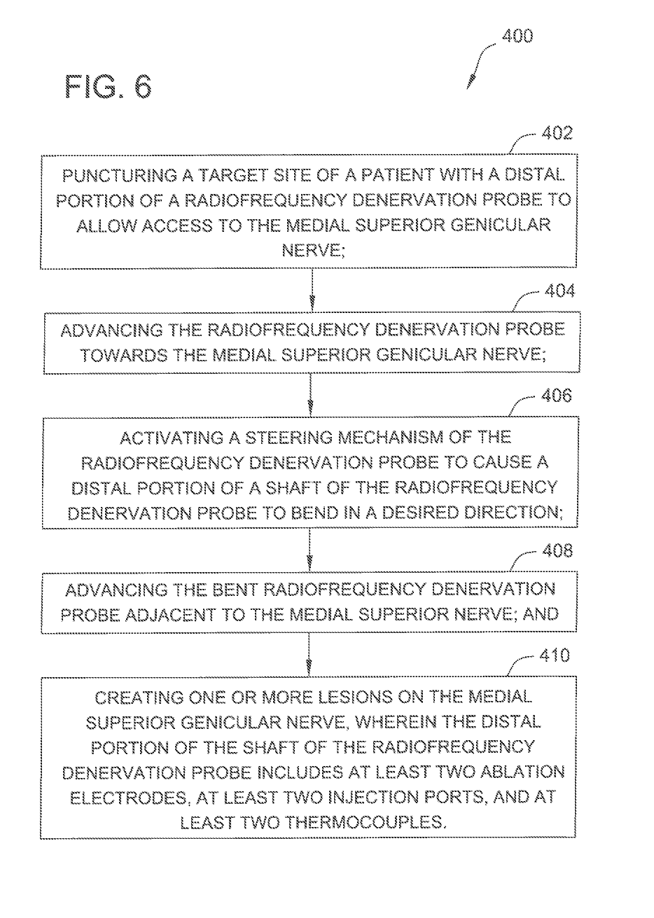

In another embodiment of the present disclosure, the steerable genicular denervation probes of the present disclosure can be used in a method of reducing pain associated with chronic knee osteoarthritis. This method includes puncturing a target site of a patient with a distal portion of a radiofrequency denervation probe to allow access to the medial superior genicular nerve. After puncturing, the radiofrequency denervation probe is advanced towards the medial superior genicular nerve and a steering mechanism of the radiofrequency denervation probe is activated to cause a distal portion of a shaft of the radiofrequency denervation probe to bend in a desired direction. The bent radiofrequency denervation probe is then advanced adjacent to the medial superior genicular nerve and one or more lesions are created on the superior genicular nerve. In many embodiments, the radiofrequency denervation probe includes at least two ablation electrodes, at least two injection ports, and at least two thermocouples, although other configurations as described herein are also within the scope of the present disclosure. Also, although this specific embodiment is directed at the medial superior genicular nerve, it is within the scope of the present disclosure to create one or more lesions on any or all of the genicular nerves as discussed herein. FIG. 6 is a flow chart of one embodiment of a method 400 for reducing the pain associated with chronic knee osteoarthritis. Method 400 includes puncturing 402 a target site of a patient with a distal portion of a radiofrequency denervation probe to allow access to the medial superior genicular nerve; advancing 404 the radiofrequency denervation probe towards the medial superior genicular nerve; activating 406 a steering mechanism of the radiofrequency denervation probe to cause a distal portion of a shaft of the radiofrequency denervation probe to bend in a desired direction; advancing 408 the bent radiofrequency denervation probe adjacent to the medial superior nerve; and creating 410 one or more lesions on the medial superior genicular nerve, wherein the distal portion of the shaft of the radiofrequency denervation probe includes at least two ablation electrodes, at least two injection ports, and at least two thermocouples.

In yet another embodiment of the present disclosure, the steerable genicular nerve denervation probes may be utilized for the denervation of the three branches of the genicular nerve for knee joint denervation. In this embodiment, a patient or subject is placed in a supine position with a support (such as a pillow) under the popliteal fossa to alleviate potential discomfort. An anteroposterior fluoroscopic view of the knee joint is then obtained and the skin and soft tissue around the knee are anesthetized with lidocaine or another suitable agent. After the lidocaine is applied, the genicular nerve denervation probe is advanced percutaneously (from the anterior, with insertion medial to the lateral most aspect of the bone) towards the targeted genicular nerve until the distal tip of the probe passes the lateral-most aspect of the bone (when positioning for lateral targets; medial-most aspect of the bone when positioning for medial targets). Once the advancement is complete, a lateral fluoroscopic view may be done to confinn that the distal tip of the probe is at the medial aspect of the bone from the lateral viewing standpoint. After this is complete, the steering mechanism of the probe is activated to create a 15 to 20 degree of curvature in the distal portion of the probe and the probe is advanced to the final position (about 10 millimeters in most cases).

After the denervation probe is advanced to its final position, another anteroposterior fluoroscopic view is taken to confirm the proximity of the probe to the bone and the placement of the radiopaque and thermocouple directional indicators adjacent to the bone. A lateral view is also taken to confirm adequate anterior to posterior coverage of the bone with the active portion (electrodes) of the probe. Once these views have been taken and confirmed, sensory and motor testing may be performed and a local anesthetic is injected through the probe into the site and allowed to sufficiently absorb. Once the anesthetic has been absorbed, the desired lesions are created (using a preset algorithm or customized lesion creation protocol) and additional anesthetic or steroids may be injected. After injection, the probe is removed and the insertion site is wrapped and bandaged as necessary.

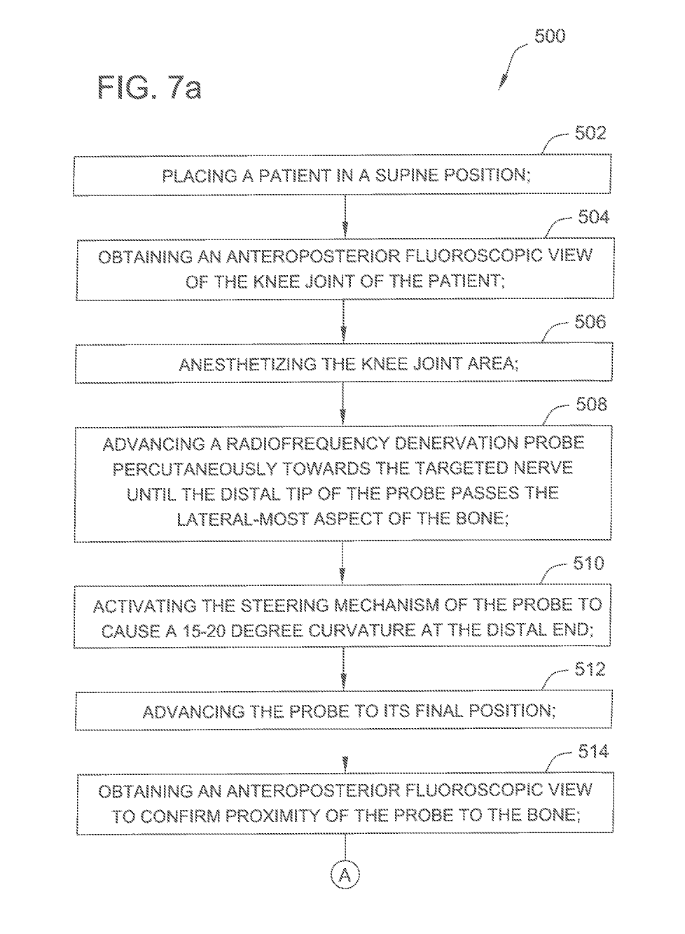

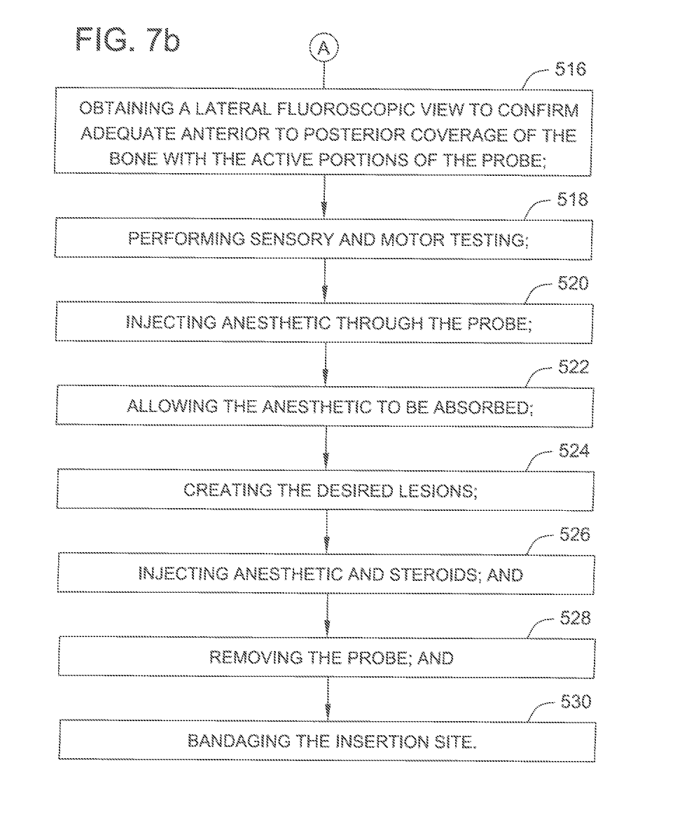

FIGS. 7a and 7b are flow charts of one embodiment of a method of denervating three branches of the genicular nerve for knee joint intervention. Method 500 includes placing 502 a patient in a supine position; obtaining 504 an anteroposterior fluoroscopic view of the knee joint of the patient; anesthetizing 506 the knee joint area; advancing 508 a radiofrequency denervation probe percutaneously towards the targeted nerve until the distal tip of the probe passes the lateral-most aspect of the bone; activating 510 the steering mechanism of the probe to cause a 15-20 degree curvature at the distal end; advancing 512 the probe to its final position; obtaining 514 an anteroposterior fluoroscopic view to confirm proximity of the probe to the bone; obtaining 516 a lateral fluoroscopic view to confirm adequate anterior to posterior coverage of the bone with the active portions of the probe; performing 518 sensory and motor testing; injecting 520 anesthetic through the probe; allowing 522 the anesthetic to be absorbed; creating 524 the desired lesions; injecting 526 anesthetic and steroids; and removing 528 the probe and bandaging 530 the insertion site.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. A method of treating chronic pain in the knee, the method comprising:
puncturing a target site of a patient with a distal portion of a radiofrequency denervation probe to allow access to at least one genicular nerve;
advancing the radiofrequency denervation probe towards the at least one genicular nerve;
activating a steering mechanism of the radiofrequency denervation probe to cause a distal portion of a shaft of the radiofrequency denervation probe to bend in a desired direction;
advancing the bent radiofrequency denervation probe adjacent to the at least one genicular nerve; and
creating one or more lesions on the at least one genicular nerve;
wherein the distal portion of the shaft of the radiofrequency denervation probe includes at least one directional ablation electrode.

2. The method of claim 1, further comprising advancing the radiofrequency denervation probe towards the at least one genicular nerve until the distal portion of the radiofrequency denervation probe has passed a lateral-most aspect of a bone adjacent the at least one genicular nerve prior to activating the steering mechanism.

3. The method of claim 1, further comprising activating the steering mechanism of the radiofrequency denervation probe to cause the bend to have a curvature of from about 15 degrees to about 20 degrees.

4. The method of claim 1, further comprising obtaining a fluoroscopic view of the target site prior to puncturing the target site.

5. The method of claim 4, further comprising obtaining a fluoroscopic view of the target site after advancing the bent radiofrequency denervation probe adjacent to the at least one genicular nerve.

6. The method of claim 1, further comprising treating the distal portion of the radiofrequency denervation probe with a silicone lubricant prior to puncturing the target site.

7. The method of claim 1, wherein the steering mechanism is configured to bend uni-directionally.

8. The method of claim 1, wherein the radiofrequency probe includes at least two directional ablation electrodes and at least two injection ports.

9. The method of claim 1, wherein the radiofrequency denervation probe additionally includes at least one thermocouple.

10. A method of reducing the pain associated with chronic knee osteoarthritis, the method comprising:
puncturing a target site of a patient with a distal portion of a radiofrequency denervation probe to allow access to the medial superior genicular nerve;
advancing the radiofrequency denervation probe towards the medial superior genicular nerve;
activating a steering mechanism of the radiofrequency denervation probe to cause a distal portion of a shaft of the radiofrequency denervation probe to bend in a desired direction;
advancing the bent radiofrequency denervation probe adjacent to the medial superior genicular nerve; and
creating one or more lesions on the medial superior genicular nerve;
wherein the distal portion of the shaft of the radiofrequency denervation probe includes at least two ablation electrodes, at least two injection ports, and at least two thermocouples.

11. The method of claim 10, further comprising activating the steering mechanism of the radiofrequency denervation probe to cause the bend to have a curvature of from about 15 degrees to about 20 degrees.

12. A genicular nerve denervation probe comprising:
a handle including a steering mechanism;
a catheter shaft having proximal portion and a distal portion, wherein the distal portion is configured for initial percutaneous advancement, and wherein the catheter shaft is configured to allow the distal portion to bend uni-directionally at a predetermined position using the steering mechanism; and
at least one directional ablation electrode located on the distal portion of the catheter shaft.

13. The genicular nerve denervation probe of claim 12, wherein the distal portion is configured to bend to create an angle of from about 15 degrees to about 20 degrees.

14. The genicular nerve denervation probe of claim 12, further comprising at least one thermocouple.

15. The genicular nerve denervation probe of claim 12, further comprising at least one radiopaque marker.

16. The genicular nerve denervation probe of claim 12, further comprising insulation configured about the at least one directional ablation electrode.

17. The genicular nerve denervation probe of claim 12, wherein the distal portion includes a sharpened tip to allow for initial percutaneous advancement.

18. The genicular nerve denervation probe of claim 12, comprising two directional ablation electrodes, three injection ports, and two thermocouples.

19. The genicular nerve denervation probe of claim 18, further comprising insulation about the two directional ablation electrodes.

20. A method of treating chronic pain in the knee, the method comprising:
puncturing a target site of a patient with a distal portion of a radiofrequency denervation probe to allow access to at least one genicular nerve;
advancing the radiofrequency denervation probe towards the at least one genicular nerve;
activating a steering mechanism of the radiofrequency denervation probe to cause a distal portion of a shaft of the radiofrequency denervation probe to bend in a desired direction; wherein the distal portion of the shaft of the radiofrequency denervation probe includes at least a proximal directional ablation electrode and a distal directional ablation electrode with respect to the bend;
advancing the bent radiofrequency denervation probe adjacent to the at least one genicular nerve;
creating a first bipolar lesion using the proximal directional ablation electrode and the distal directional ablation electrode simultaneously;
creating a second monopolar lesion using the proximal directional ablation electrode; and
creating a third monopolar lesion using the distal directional ablation electrode.
